(19) United States
(12) Patent Application Publication
(10) Pub. No.: US 2006/0057653 A1
(43) Pub. Date: Mar. 16, 2006

(54) Detection of microorganisms with holographic sensors

(76) Inventors: Christopher Robin Lowe, Cambridge (GB); Colin Alexander Bennett Davidson, Cambridge (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/520,221

(22) PCT Filed: Jul. 9, 2003
(86) PCT No.: PCT/GB03/02958

§ 371 Date: Jul. 27, 2005

(30) Foreign Application Priority Data
Jul. 9, 2002 (GB) 0215878.0

(51) Int. Cl. G01N 33/554; C12Q 1/04; C12M 1/34; G01N 33/569

(52) U.S. Cl. 435/7.32; 435/34; 435/287.1

(57) ABSTRACT

A method for the detection of a cell comprises immobilizing the cell in a device also containing a sensor, and introducing a growth medium, wherein the sensor is sensitive to a product of the cell's growth; and detecting any change in an optical characteristic of the sensor. A device suitable for use in the invention comprises a chamber including a sensor, inlets for sample and for a growth medium, and means for immobilizing an antibody in the chamber or elsewhere in the device that provides a fluidic link with the sensor.

### FIELD OF THE INVENTION

This invention relates to the detection of cells, e.g. using a holographic sensor.

### BACKGROUND TO THE INVENTION

Rapid identification of cells, in particular pathogenic cells, is of vital importance in diagnostics and biodefence. Whilst there are a number of competing technologies available to aid in this process, such as ELISA and PCR, the definitive identification of a microbial pathogen is still a time-consuming, laboratory-based procedure.

ELISA kits for the detection of agents such as Bacillus anthracis are available. These kits are highly specific to the target organism, showing no cross-reaction with closely related Bacillus species. They are, however, somewhat insensitive, requiring in the order of 10,000 cells, in order to avoid false negatives; this quantity of cells is somewhat more than a human infective dose of a microbe such as Bacillus anthracis.

PCR technology provides a fast, accurate and rapid means for determining the identity of a disease-causing agent. Unfortunately, this technology is sensitive to environmental contamination, meaning that sample pre-treatment is necessary in many instances. This technology is also expensive and requires highly trained personnel.

Neither of these methods is readily compatible with conventional microbiology techniques. While they may be used in some circumstances to determine the identity of a microbe in a large or pure sample, they do not readily lend themselves to direct comparison with laboratory assays in which cells are cultured and identified using classical microbiological methodologies. Nor do they provide a means for capturing viable cells for definitive identification.

Holographic sensors may be used for the detection of a variety of analytes. WO-A-9526499 discloses a holographic sensor, based on a volume hologram. This sensor comprises an analyte-sensitive matrix having an optical transducing structure disposed throughout its volume. Because of this physical arrangement of the transducer, the optical signal generated by the sensor is very sensitive to volume changes or structural rearrangements taking place in the analyte-sensitive matrix as a result of interaction or reaction with the analyte.

### SUMMARY OF THE INVENTION

According to one aspect of the invention, a method for the detection of a cell comprises immobilising the cell in a device also containing an optical sensor, and introducing a growth medium. The sensor is sensitive to a product of the cell's growth, and a change in an optical characteristic of the sensor is detected. Preferably, the cell is immobilised using an antibody.

Another aspect of the invention is a device suitable for use in a method of the invention, the device comprising a chamber including a sensor and a growth medium, and an inlet for a sample and optionally comprising means for immobilising an antibody in the chamber or elsewhere in the device that provides a fluidic link with the sensor. The device preferably comprises a container comprising a buffer solution and an outlet leading to the sample inlet of the chamber. An antibody may be immobilised on a wall of the chamber, or on a magnetic particle.

The invention allows rapid, accurate identification of the target organism, with the specificity of ELISA technology. Detection can be made under a wide range of conditions, e.g. at sub-infectious concentrations. A device of the invention may be simple to operate and compatible with standard laboratory techniques. By directly interfacing a device of the invention with PCR technology, full integration with laboratory-based diagnostics is possible.

### DESCRIPTION OF PREFERRED EMBODIMENTS

A cell may be held in the chamber by the growth medium, and this may be sufficient particularly if the sample is not mixed. A cell may be immobilised by my suitable means, for example using an agent such as an antibody. The cell may then be cultured in situ, in a range of determinative microbiological growth media and in the presence of the holographic sensor. Products released into the growth media during germination may also be detected. Germination of bacterial spores, as well as subsequent growth, typically requires the presence of specific nutrients, divalent ions and a specific pH range. The requirements for germination may differ from those for outgrowth.

Upon capture, detection can be made by monitoring the activity of the cell. The sensor is “optical” in the sense that it can be observed using optics. Typically, it is a holographic sensor. A holographic sensor can be used to detect species such as biodegradative enzymes or very small changes in pH and redox potential. For example, acidic species can be detected using a pH-sensitive holographic element. As the pH changes, the holographic element undergoes a swelling or contraction, resulting in a colour change of the reflected wavelength. The sensor that is used may be of the type described in WO-A-9526499 or WO-A-9963408, the contents of which are incorporated herein by reference.

A method of the invention can be used to detect pathogens of bio-warfare Escherichia coli spp., Campylobacter spp., and bio-terrorist interest (e.g. Yersinia pestis and Francisella tularensis) as well as pathogens of interest in environmental and medical monitoring (e.g. Legionella spp. and Salmonella spp.). Other bacteria which may be detected include Listeria spp. and those of the genus Bacillus, e.g. Bacillus anthracis, Bacillus thuringiensis, Bacillus globigii, Bacillus megaterium and Bacillus subtilis.

An example of whole cell detection is that of the bacterium, Legionella pneumophilia, which is associated with Legionnaire's disease (Legionellosis) and Pontiac fever. L. pneumophilia serogroup1 is the most frequently implicated in human disease and is usually found in aqueous environments. The bacteria survive in low numbers in routine water treatment and reproduce to high numbers in warm, stagnant water. The bacterium may be immobilised with an appropriate monoclonal antibody. For example, a purified IgG3 class mouse monoclonal antibody that recognises the lipopolysaccharide antigen of heat-resistant L. pneumophilia serogroup 1 is commercially available.

The immobilised cell is then cultured, and a metabolic product detected. One approach is to use a pH-sensitive hologram; L. pneumophilia hydrolyses hippuric acid to generate benzoic acid, producing a swelling and colour change of the hologram. A similar approach can be used to detect the ability of the organism to hydrolyse penicillins. Any additional penicillin will be hydrolysed by the intrinsic β-lactamase of L. pneumophilia, and the resulting penicilloic acid may be detected using a pH-sensitive hologram. An alternative approach exploits the fact that L. pneumophilia has endogenous oxidase activity, generating hydrogen peroxide from appropriate substrates. Hydrogen peroxide reacts with iodine to generate iodide ions. In the presence of iodine, a holographic sensor comprising silver grains can be used to detect hydrogen peroxide since any iodide ions formed react with silver to form silver iodide. Holograms can respond to added and enzymatically generated hydrogen peroxide via this mechanism.

As indicated above, a pH sensor may be used. This will allow detection of a pH change associated with nutrient source utilisation, e.g. of carbohydrates in bacteria.

A starch-based holographic sensor may be used to detect cells which generate amylase as a growth product; amylase causes the degradation of starch. The Bacillus genus is characterised by relatively high amylase production during growth and thus a starch-based sensor is particularly suitable.

The invention is particularly suitable for the detection of spores, and to monitor their germination.

For example, spores of the Bacillus genus typically release Ca²⁺ (e.g. in the form of the diplicolinic acid salt thereof) during germination. Calcium ions bind to a polyHEMA-polyMIDA holographic support medium inducing concentration of the medium and a shift in the replay wavelength. By using such a support medium, germination of Bacillus spores can be detected.

Germination can also be detected by monitoring the activity of spore proteases. The cell wall of a spore typically comprises a thick peptinoglycan layer which can be degraded by the activation of specific endogenous enzymes. By incorporating an appropriate peptinoglycan matrix in a holographic sensor, these enzymes can be detected.

A device of the invention comprises an inlet (such as a flip-top well) into which a test sample is placed. The sample may be present in or on a swab which can be placed at or near to the inlet. Fluid may be passed through the swab, collecting the sample and transferring it to the growth chamber. The sample is preferably transferred by a fluid (e.g. a buffer solution) to a growth chamber comprising the sensor and, preferably an immobilising agent (e.g. an antibody), which captures the organism prior to the addition of growth medium. Cells may also be immobilised using a suitable filter. Antibodies may be immobilised on one or more walls of a chamber or on magnetic particles upstream of the growth chamber; if desired, the particles may be transferred to the chamber using a magnet present in the device. Alternatively, a cell may be immobilised upstream of the sensor, provided that the two have a fluidic link, i.e. that a product of the cell can flow into contact with the sensor. A growth medium is then fed into the device, and the growth of any specifically bound organisms can be detected, by observation of the sensor. A change of a property of the hologram can be observed using any suitable apparatus, e.g. as described in WO-A-9526499.

A device of the invention preferably comprises multiple cell capture chambers. The test sample may be mixed with a basal growth medium, which can be added to a series of fermentation wells, each containing dried carbon and/or nitrogen sources and a holographic sensor. Should magnetic particles be used, then each cell is preferably backed by a magnetic strip to capture the particles on which the test organism is immobilised. The device may further comprise a well downstream from the growth chamber, to collect excess and waste samples.

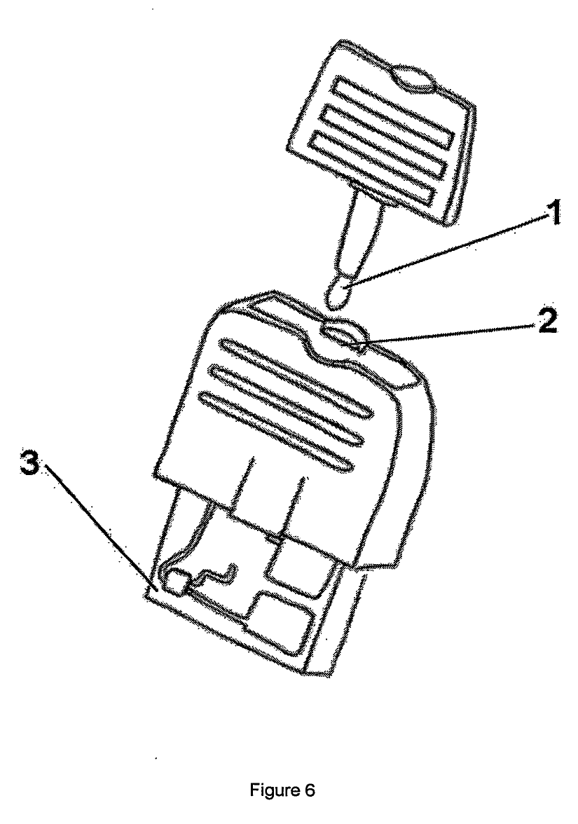

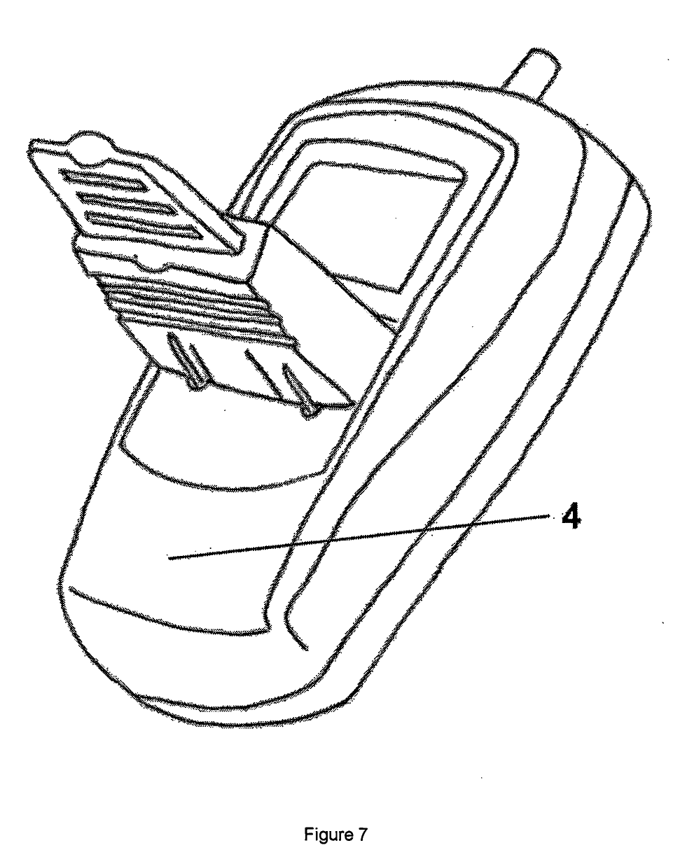

An embodiment of a device of the invention will now be described by way of example with reference to FIGS. 6 and 7. FIG. 6 is a perspective view of such a device, and shows a swab 1 mounted on a member, insertable into a unit having an inlet 2 and including a fluidic array at 3. In use, a sample collected on the swab can be transferred by operating a pump (not shown) to the fluidic array 3 which comprises one or more growth chambers connected by fluidic channels.

The device is designed so that it can be directly inserted into an optical reader; FIG. 7 shows the device of FIG. 6 inserted into a reader 4. The fluidic array is exposed in the body of the reader allowing one or more measurements (e.g. holographic replay wavelength) to be taken.

The invention will now be described by way of example, with reference to the accompanying drawings.

### EXAMPLE 1

Bacillus subtilis was detected in microbial culture. A metabolic product of the bacterium is protease, which degrades a gelatin-based holographic sensor. As the gelatin support medium degrades, it becomes increasingly spongy and expands.

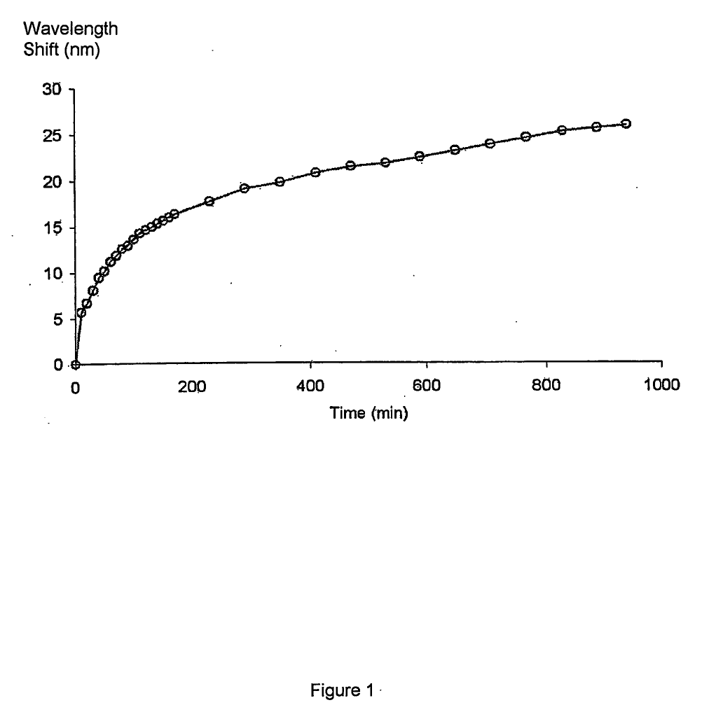

Mid-exponential phase culture (in nutrient broth) was inoculated into a cuvette containing the hologram, and a reflection spectrometer used to measure the peak wavelength at 10 minute intervals over 15 hours at 30° C. A positive result for protease was shown by the peak wavelength undergoing a red-shift. FIG. 1 shows the red-shift of the peak wavelength of reflection over the 15 hour period.

### EXAMPLE 2

Bacillus megaterium was detected in microbial culture. During germination, the bacterium releases Ca²⁺ (bound to dipicolinic acid). Ca²⁺ binds to a polyHEMA-MIDA holographic support medium, inducing a concomitant contraction of the polymer and a shift in replay wavelength.

A holographic sensor compound of 10 and 12 mole % MIDA in polyHEMA was equilibrated in nutrient broth. Bacillus megaterium spores were then added at a concentration of approximately 10⁸ spores/ml. A reflection spectrometer was used to measure the peak wavelength at 1 minute intervals for 50 minutes at 25° C. Any change in the optical density of the sensor was also detected, a change in optical density being indicative of germination. Changes in the optical density of the germination matrix were also detected.

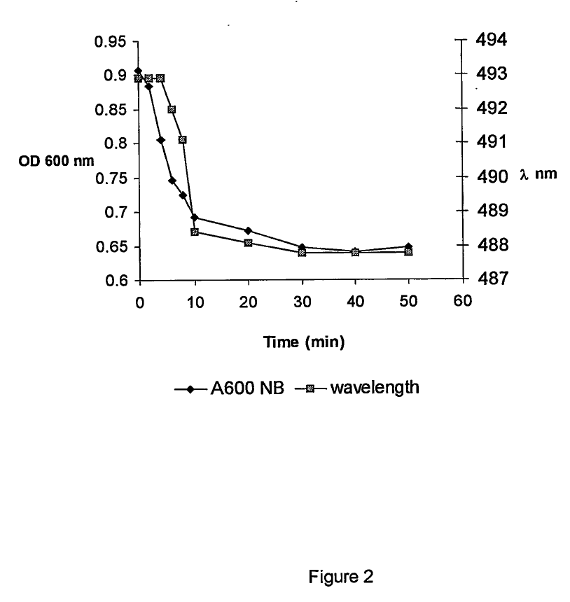

FIG. 2 is a graph of the germination response, showing the optical density (OD) and wavelength readings. The decreases in both OD and A are indicative of Ca²⁺-induced binding to of the holographic support medium. The results suggest that germination occurred within the first 10 minutes.

### EXAMPLE 3

Vegetative Bacillus megaterium was detected using a starch/acrylamide holographic sensor. The Bacillus genus is characterised by relatively high amylase production during growth; amylase degrades a starch-based holographic support medium.

A section of the sensor was equilibrated with 1800 μl of nutrient both at 30° C. 200 μl of vegetative Bacillus megaterium (cultured overnight) was then added (the cells were centrifuged and resuspended in fresh medium prior to addition to the cuvette, to remove any residual amylase). The peak wavelength of reflection of the sensor was recorded every 15 minutes for approximately 16 hours.

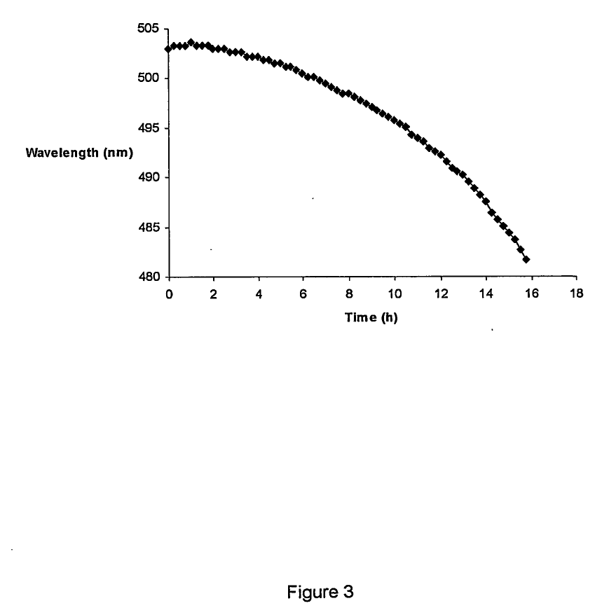

The results are shown in FIG. 3. Initially, the shift in wavelength was relatively small; however, the shift was more pronounced with time. This lag may be due to the presence of residual glucose in the holographic support medium.

### EXAMPLE 4

A holographic sensor having a support medium compound of 6% MMA co HEMA was used to detect the growth of Bacillus megaterium spores in nutrient broth. 200 μl of the spores were added (at a concentration ˜108 spores/ml) to a cuvette containing the sensor, the nutrient broth and also a pH probe. The holographic replay wavelength and pH were measured over approximately 125 minutes.

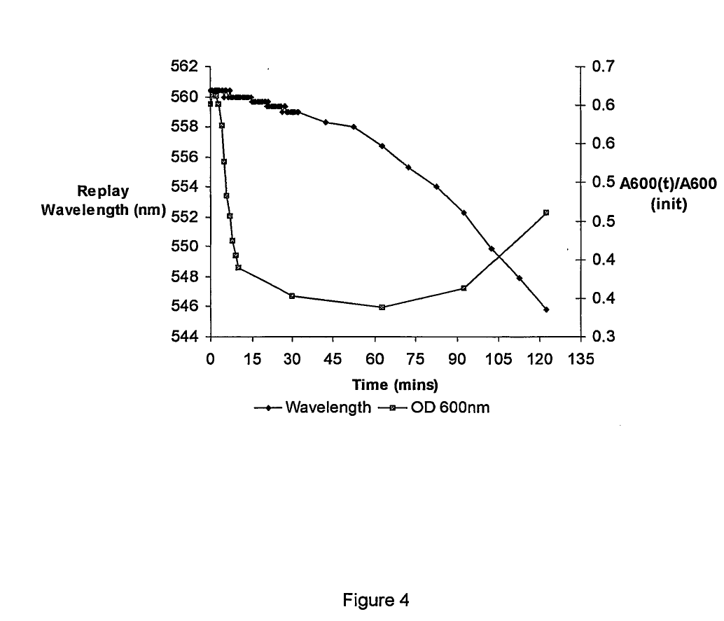

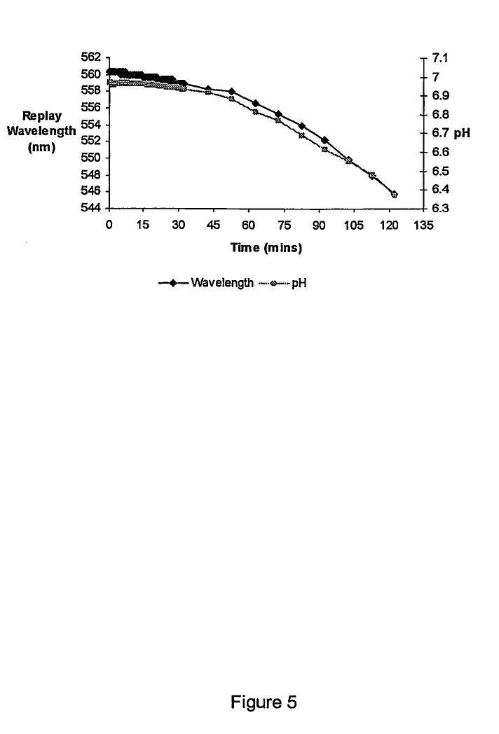

Results are shown in FIGS. 4 and 5, i.e. respective graphs showing germination response. The correlation between A and pH is excel lent, accurately reflecting the extent of germination.

1. A method for the detection of a cell, wherein said method comprises immobilising the cell in a device also containing a sensor, and introducing a growth medium, wherein the sensor is sensitive to a product of the cell's growth; and detecting any change in an optical characteristic of the sensor.

2. The method according to claim 1, wherein the cell is immobilised on a magnetic particle.

3. The method according to claim 1, wherein the cell is a spore cell.

4. The method according to claim 1, wherein the cell is a bacterial cell.

5. The method according to claim 4, wherein the bacterium is selected from the group consisting of Bacillus anthracis, Bacillus globigii, Bacillus subtillis, Bacillus megaterium, Legionella pneumophilia, Francisella tularensis, Yersinia pestis, Salmonella spp., E. coli spp., Listeria spp., Bacillus thuringiensis and Campylobacter spp.

6. The method according to claim 1, wherein the cell is immobilised by means of an antibody.

7. The method according to claim 1, wherein the sensor is a holographic sensor.

8. A device suitable for the detection of a cell by a method comprising immobilising the cell in a device that also contains a sensor, and introducing a growth medium, wherein the sensor is sensitive to a product of the cell's growth; and detecting any change in an optical characteristic of the sensor;
wherein said device comprises a chamber including a sensor and a growth medium, and an inlet for a sample.

9. The device according to claim 8, which further comprises means for immobilising an antibody in the chamber or elsewhere in the device that provides a fluidic link with the sensor.

10. The device according to claim 9, wherein the antibody is immobilised on a wall of the chamber.

11. The device according to claim 9, which additionally comprises the antibody immobilised on a magnetic particle, and the said means can provide a magnetic field.

12. The device according to claim 8, further comprising a container including a buffer solution, in connection with the sample inlet.

13. The device according to claim 8, which comprises a series of said chambers.

14. The device according to claim 8, wherein the sensor is a holographic sensor.
